(19) United States
(12) Patent Application Publication
(10) Pub. No.: US 2021/0386562 A1
(43) Pub. Date: Dec. 16, 2021

(54) UPPER LIMB PROSTHESES

(71) Applicant: The Board of Regents of the University of Nebraska, Lincoln, NE (US)

(72) Inventors: Jorge Zuniga, Walton, NE (US); Jean Peck, Omaha, NE (US); Walker Arce, Omaha, NE (US); Rakesh Srivastava, Hastings, NE (US); James Pierce, Omaha, NE (US); Keaton Young, Omaha, NE (US)

(21) Appl. No.: 17/286,382

(22) PCT Filed: Oct. 18, 2019
(86) PCT No.: PCT/US2019/056902

§ 371 Date: Jul. 29, 2021

Related U.S. Application Data
(60) Provisional application No. 62/750,329, filed on Oct. 25, 2018.
(60) Provisional application No. 62/747,935, filed on Oct. 19, 2018.

(51) Int. Cl. A61F 2/58; A61F 2/68

(52) U.S. Cl. CPC A61F 2/586; A61F 2002/543; A61F 2/68

(57) ABSTRACT

An upper limb prosthetic device may comprise a hybrid-drive prosthetic device that includes one or more actuators fixed to a first arm portion and one or more cables extending from the one or more actuators along one or more cable paths defined through the first arm portion and a second arm portion to a terminal device/device assembly, where the one or more cables are configured to be actuated by the one or more actuators and are further configured to be actuated by pivoting the second arm portion toward the first arm portion, Alternatively, or additionally, the upper limb prosthetic device may comprise a modular prosthetic device that includes a modular terminal device assembly with removably coupled first, second, and third modular links configured to control the respective yaw, pitch, and roll of an end effector (e.g a prosthetic hand, a tool, an instrument or any other attachment).

### CROSS-REFERENCE TO RELATED APPLICATIONS

The present application claims the benefit of U.S. Provisional Application No. 62/747,935, filed Oct. 19, 2018, and titled “Hybrid-Drive Prosthesis,” which is herein incorporated by reference in its entirety. The present application also claims the benefit of U.S. Provisional Application No. 62/750,329, filed Oct. 25, 2018, and titled “Modular Task-Specific Prosthesis.” which is herein incorporated by reference in its entirety.

### BACKGROUND

Traditional electronically-powered upper limb prostheses have two common downfalls. They tend to be heavy, especially toward the distal end (e.g., hand) of the prosthesis. They also tend to have very limited manual, body-powered control (if any). Consequently, there is need for upper limb prostheses with improved weight distribution and usability.

Generalized body-powered upper limb prostheses are effective at gross movements, such as full-hand grasping, but are not robust in the performance of more morphologically or technically challenging tasks. For this reason, existing prostheses are generally not intended for specific activities. Additionally, those existing devices which are activity-specific cannot be easily adapted to other specific tasks. Consequently, there is also a need for upper limb prostheses with improved adaptability for specific tasks.

### SUMMARY

A hybrid-drive prosthetic device is disclosed. In embodiments, the hybrid-drive prosthetic device includes at least a first arm portion and a second arm portion. The first arm portion is configured to at least partially surround a portion of a limb. The second arm portion is pivotally coupled to the first arm portion. The second arm portion includes a socket configured to receive a distal end of the portion of the limb and has a terminal device (e.g., a prosthetic hand or any other end-effector(s)) coupled to a distal end of the second arm portion. In embodiments, the hybrid-drive prosthetic device further includes one or more actuators fixed to the first arm portion and one or more cables extending from the one or more actuators along one or more cable paths defined through the first arm portion and the second arm portion to the terminal device. The one or more cables may be configured to control one or more components (e.g., actuatable components/portions) of the terminal device. In embodiments, the one or more cables are configured to be actuated by the one or more actuators and are further configured to be actuated by pivoting the second arm portion toward the first arm portion. This allows for semi-automated (human-assisted) actuation of components in the terminal device when the actuators are not capable of supplying enough torque to complete a task.

A modular prosthetic device is also disclosed. In embodiments, the modular prosthetic device includes at least a first arm portion and a second arm portion. The first arm portion is configured to at least partially surround a portion of a limb. The second arm portion is pivotally coupled to the first arm portion. The second arm portion includes a socket configured to receive a distal end of the portion of the limb and has a modular terminal device assembly removably coupled to a distal end of the second arm portion. In embodiments, the modular terminal device assembly includes a first modular link, a second modular link, and a third modular link. The first modular link is removably coupled to the second arm portion and is configured to pivot about a first axis to control yaw (e.g., abduction/adduction or radial/ulnar deviation) of an end effector (e.g., a prosthetic hand, a tool, an instrument, or any other attachment). The second modular link is removably coupled to the first modular link and is configured to pivot about a second axis to control pitch (e.g., flexion/extension) of the end effector. The third modular link is removably coupled to the second modular link and is configured to rotate about a third axis to control roll (e.g., pronation/supination) of the end effector. One or more of the modular links may be removed (e.g., detached) from the modular terminal device assembly to allow for utilization of different end effectors.

A hybrid-drive modular prosthetic device is also disclosed. In embodiments, the hybrid-drive modular prosthetic device includes at least a first arm portion and a second arm portion. The first arm portion is configured to at least partially surround a portion of a limb. The second arm portion is pivotally coupled to the first arm portion. The second arm portion includes a socket configured to receive a distal end of the portion of the limb and has a modular terminal device assembly removably coupled to a distal end of the second arm portion. In embodiments, the modular terminal device assembly includes a first modular link, a second modular link, and a third modular link. The first modular link is removably coupled to the second arm portion and is configured to pivot about a first axis to control yaw (e.g., abduction/adduction or radial/ulnar deviation) of an end effector (e.g., a prosthetic hand, a tool, an instrument, or any other attachment). The second modular link is removably coupled to the first modular link and is configured to pivot about a second axis to control pitch (e.g., flexion/extension) of the end effector. The third modular link is removably coupled to the second modular link and is configured to rotate about a third axis to control roll (e.g., pronation/supination) of the end effector. One or more of the modular links may be removed (e.g., detached) from the modular terminal device assembly to allow for utilization of different end effectors. In embodiments, the hybrid-drive modular prosthetic device further includes one or more actuators fixed to the first arm portion and one or more cables extending from the one or more actuators along one or more cable paths defined through the first arm portion and the second arm portion to the modular terminal device assembly. The one or more cables may be configured to control one or more components (e.g., actuatable components/portions/links) of the modular terminal device assembly. In embodiments, the one or more cables are configured to be actuated by the one or more actuators and are further configured to be actuated by pivoting the second arm portion toward the first arm portion. This allows for semi-automated (human-assisted) actuation of components in the modular terminal device assembly when the actuators are not capable of supplying enough torque to complete a task.

This Summary is provided solely as an introduction to subject matter that is fully described in the Detailed Description and Drawings. The Summary should not be considered to describe essential features nor be used to determine the scope of the Claims. Moreover, it is to be understood that both the foregoing Summary and the following Detailed Description are example and explanatory only and are not necessarily restrictive of the subject matter claimed.

### BRIEF DESCRIPTION OF THE DRAWINGS

The detailed description is described with reference to the accompanying figures. The use of the same reference numbers in different instances in the description and the figures may indicate similar or identical items. Various embodiments or examples (“examples”) of the present disclosure are disclosed in the following detailed description and the accompanying drawings. The drawings are not necessarily to scale. In general, operations of disclosed processes may be performed in an arbitrary order, unless otherwise provided in the claims.

FIG. 1A is a perspective view of a hybrid-drive prosthetic device, in accordance with one or more embodiments of the present disclosure.

FIG. 1B is a zoomed in view of an upper arm portion of a hybrid-drive prosthetic device, in accordance with one or more embodiments of the present disclosure.

FIG. 1C is a perspective front view of a hybrid-drive prosthetic device, in accordance with one or more embodiments of the present disclosure.

FIG. 1D is a perspective rear view of a hybrid-drive prosthetic device, in accordance with one or more embodiments of the present disclosure.

FIG. 1E is a left-side view of a hybrid-drive prosthetic device, in accordance with one or more embodiments of the present disclosure.

FIG. 1F is a right-side view of a hybrid-drive prosthetic device, in accordance with one or more embodiments of the present disclosure.

FIG. 1G is a front-end view of a hybrid-drive prosthetic device, in accordance with one or more embodiments of the present disclosure.

FIG. 1H is a rear-end view of a hybrid-drive prosthetic device, in accordance with one or more embodiments of the present disclosure.

FIG. 1I is a top view of a hybrid-drive prosthetic device, in accordance with one or more embodiments of the present disclosure.

FIG. 1J is a bottom view of a hybrid-drive prosthetic device, in accordance with one or more embodiments of the present disclosure.

FIG. 2A is a perspective view of a modular prosthetic device, in accordance with one or more embodiments of the present disclosure.

FIG. 2B is an exploded perspective view of a modular prosthetic device, in accordance with one or more embodiments of the present disclosure.

FIG. 2C is a zoomed in view of a modular terminal device assembly of a modular prosthetic device, in accordance with one or more embodiments of the present disclosure.

FIG. 2D is a perspective front view of a modular prosthetic device, in accordance with one or more embodiments of the present disclosure.

FIG. 2E is a perspective rear view of a modular prosthetic device, in accordance with one or more embodiments of the present disclosure.

FIG. 2F is a right-side view of a modular prosthetic device, in accordance with one or more embodiments of the present disclosure.

FIG. 2G is a left-side view of a modular prosthetic device, in accordance with one or more embodiments of the present disclosure.

FIG. 2H is a front-end view of a modular prosthetic device, in accordance with one or more embodiments of the present disclosure.

FIG. 2I is a rear-end view of a modular prosthetic device, in accordance with one or more embodiments of the present disclosure.

FIG. 2J is a top view of a modular prosthetic device, in accordance with one or more embodiments of the present disclosure.

FIG. 2K is a bottom view of a modular prosthetic device, in accordance with one or more embodiments of the present disclosure.

FIG. 2L is a perspective view of a modular prosthetic device, in accordance with one or more embodiments of the present disclosure.

FIG. 3 is a perspective view of a hybrid-drive modular prosthetic device, in accordance with one or more embodiments of the present disclosure.

### DETAILED DESCRIPTION

Hybrid-drive and/or modular prostheses are described in accordance with various embodiments of this disclosure. In some embodiments, the prostheses described herein are at least partially formed by employing additive manufacturing technologies (e.g., three-dimensional (3D) printing). In addition to making the prostheses more customizable, 3D printing may allow for reduced cost and better user-experience, particularly when working with children.

Children's prosthetic needs are complex due to their small size, constant growth, and psychosocial development. Socio-economical background and financial resources play a crucial role in prescription of prostheses for children, especially when private insurance and public funding are insufficient. Electric-powered (e.g., myoelectric) and mechanical (e.g., body-powered) prostheses have improved to accommodate children's needs, but their maintenance and replacement costs make access difficult for many families. Voluntary-closing upper-limb prostheses are more suitable for children and could improve gross motor development. Currently, the best cost-effective option for pediatric populations is a passive prosthetic hook; although functional, these devices have a high rejection rate, in part due to weight, cost and low visual appeal. Most clinically-recommended prostheses do not adapt to the typical growth of children's limbs and require regular visits to health care providers for adjustments or replacement, which may ultimately lead to device abandonment.

However, advancements in computer-aided design software and additive manufacturing techniques (e.g., 3D printing), offer the possibility of designing, printing, and fitting prosthetic hands and other assistive devices at a relatively low cost. Studies have demonstrated that low cost prosthetic hands, arms and shoulders with practical and easy fitting procedures may be performed remotely. Importantly, in children the durability of the 3D printed prostheses is challenged continuously due to their activity levels and outgrowth of the prostheses. Therefore, the cost effectiveness of 3D printing makes repairs and upgrades of prostheses substantially more affordable. In general, previous publications have presented different aspects of the development of 3D printed prostheses for children, and the consensus is that 3D printing is a promising manufacturing method for the development of these devices.

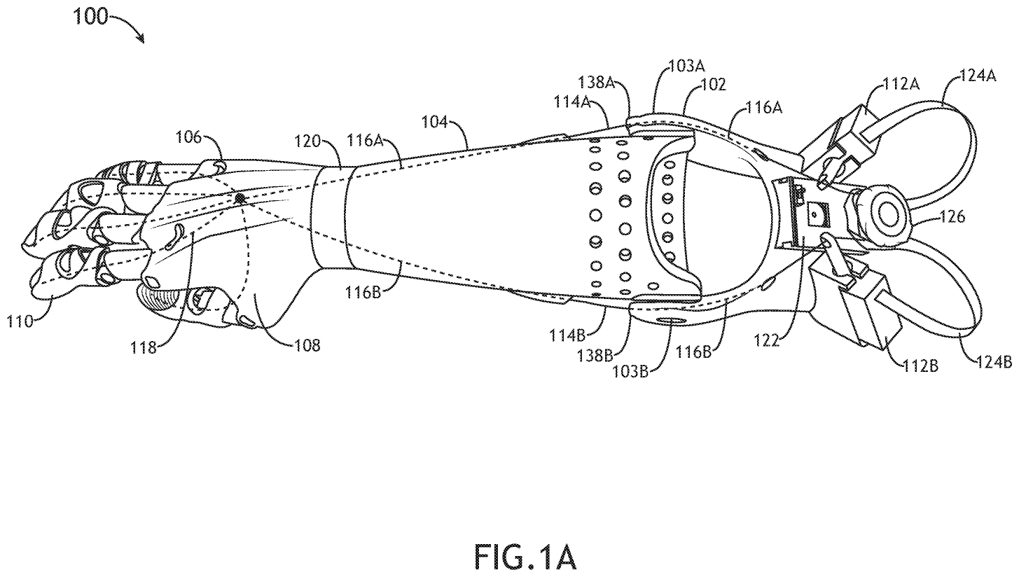

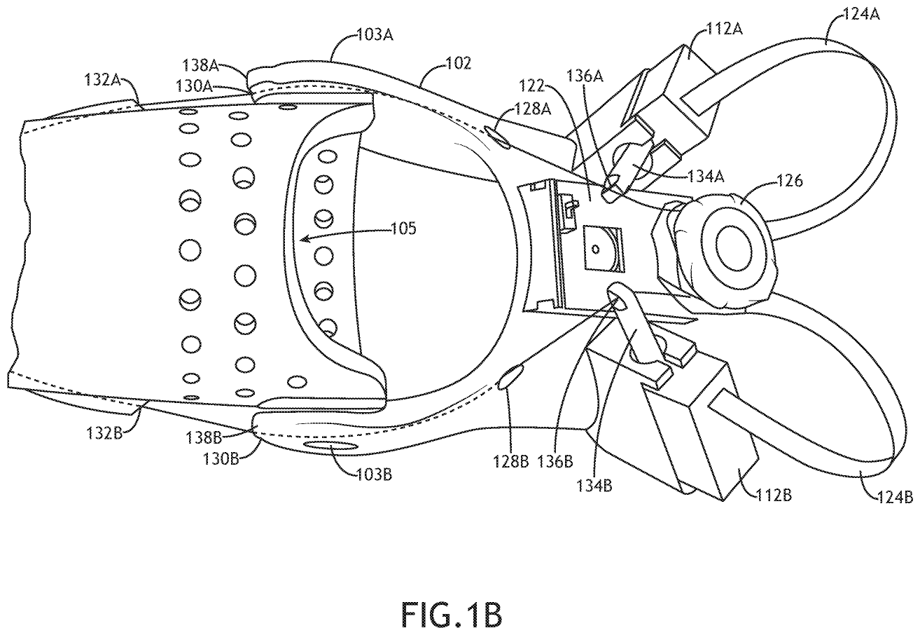

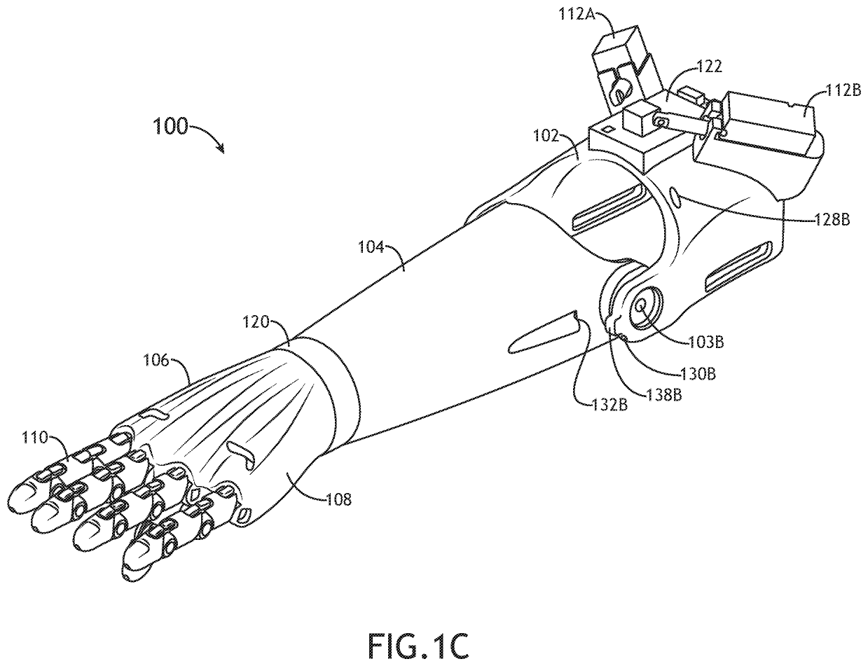

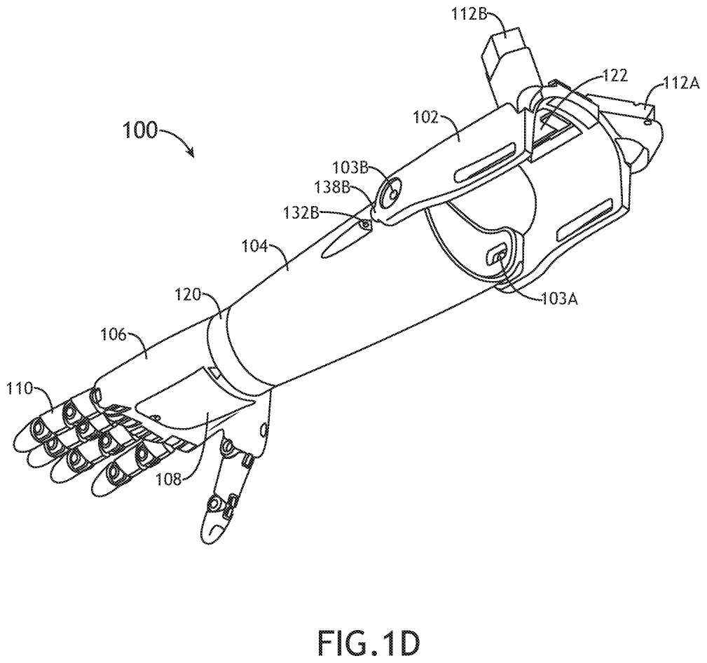

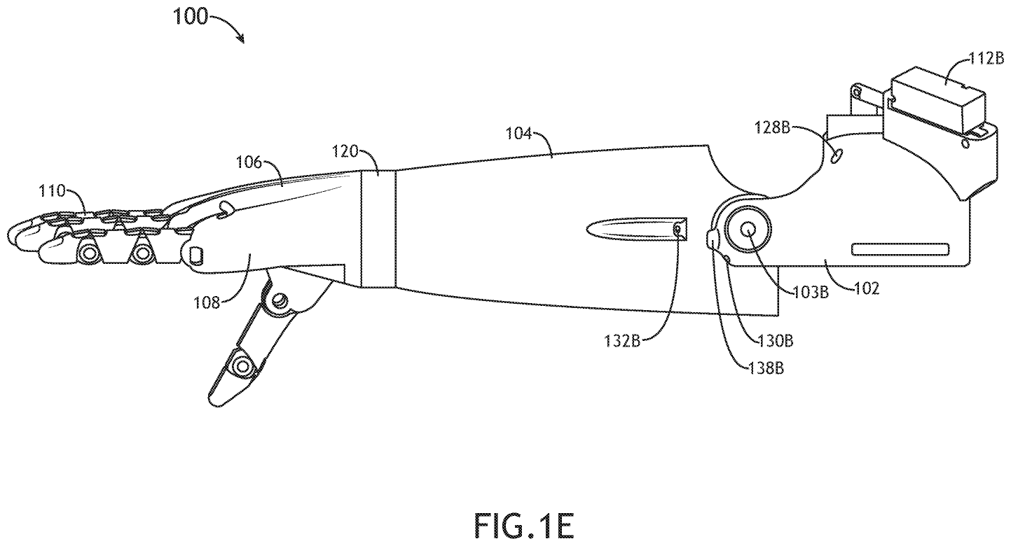

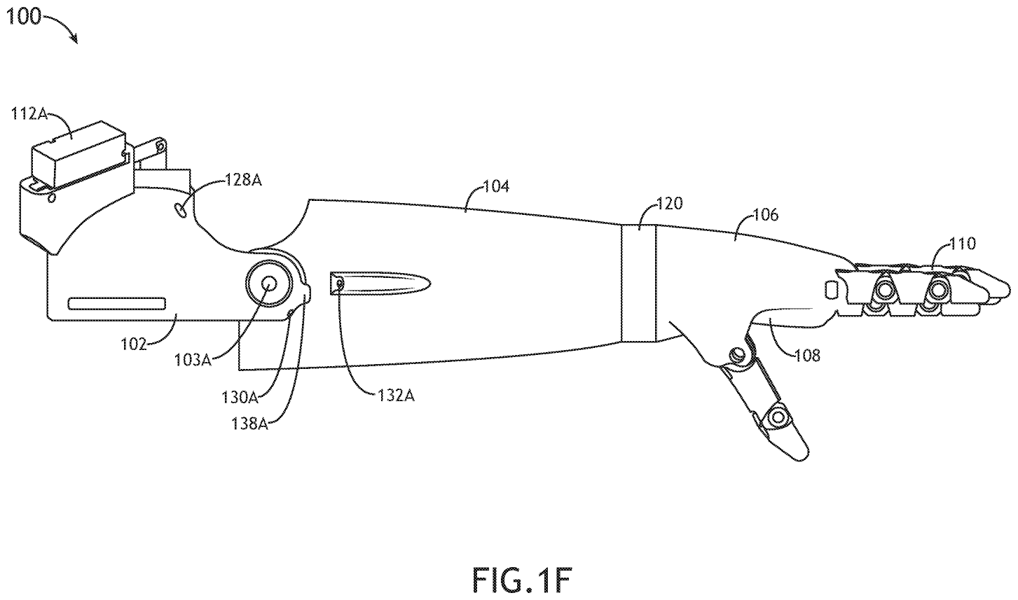

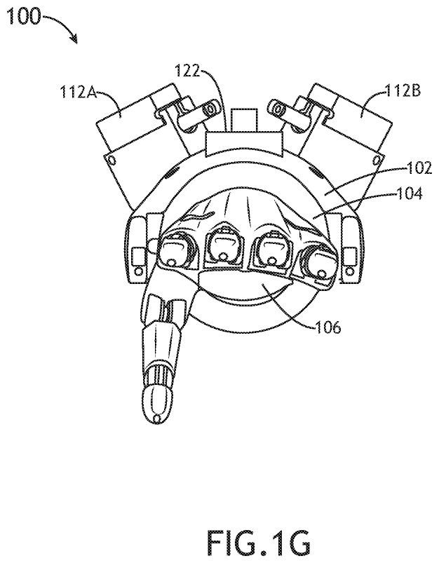

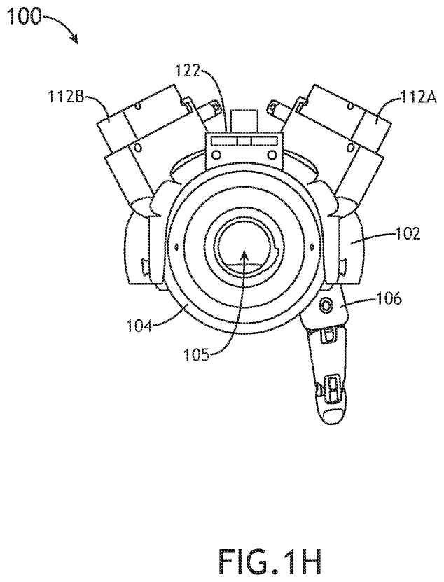

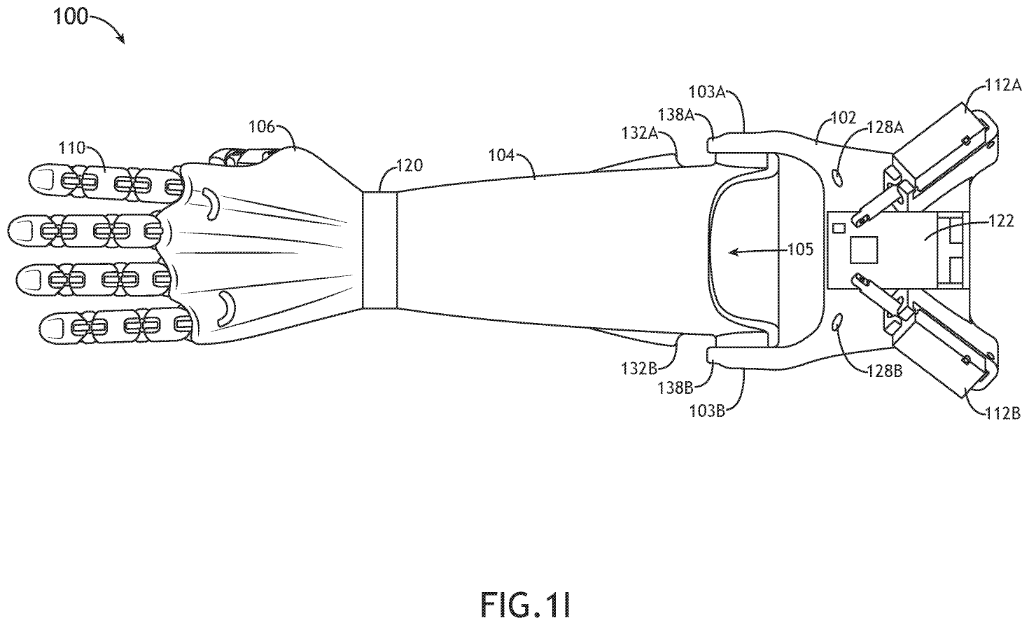

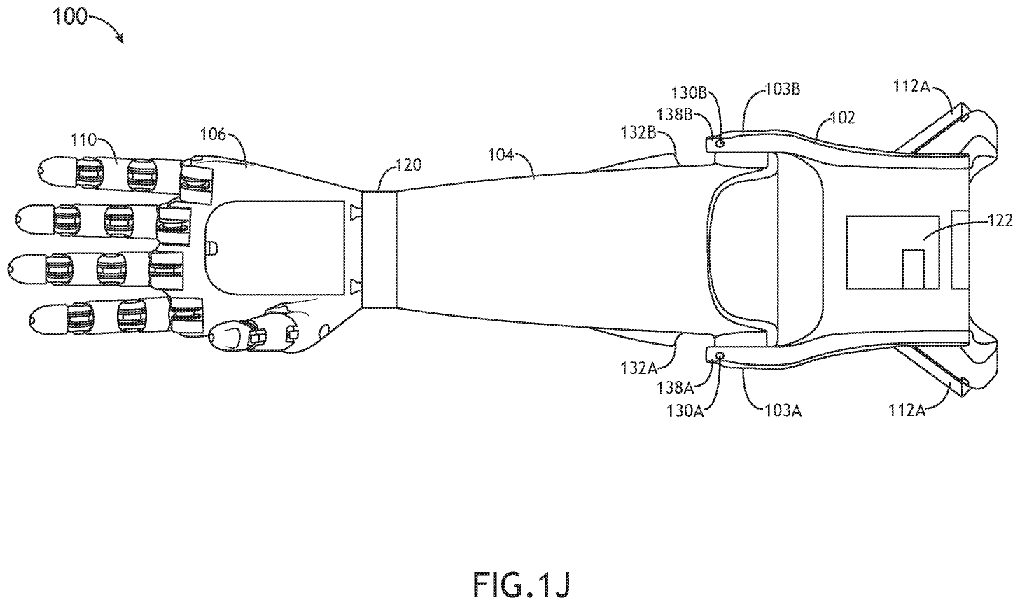

Referring now to FIGS. 1A through 1J, various embodiments of this disclosure are directed to a hybrid-drive prosthetic device 100. The hybrid-drive prosthetic device 100 strategically combines body-powered and electronically-driven motor actuation. The hybrid-drive prosthetic device 100 has benefits over current electronically-powered technology in that it is light weight, ergonomic, permits body-powered control, and is less expensive. The hybrid-drive prosthetic device 100 may provide upper-limb affected individuals with an alternative product that contains improved functionality over current body-powered devices combined with decreased muscle fatigue and cost of current electronically-powered devices.

Traditional electronically-powered upper limb prostheses have two common downfalls. They tend to be heavy, especially toward the distal end (e.g., hand) of the prosthesis. They also tend to have very limited manual, body-powered control (if any). To solve these universal issues, a hybrid-drive prosthesis design has been developed. Utilizing a body-powered prosthetic base, manual control is still entirely possible (and encouraged). This allows for the development of healthy and strong muscles in children who would normally abandon the use of their affected limb, which leads to long-term weakness and even postural problems like scoliosis. To reduce weight and enhance ease of use, fewer (and lighter weight) motors may be used. For instance, rather than developing all of the force needed to grip objects, the motors in this hybrid design may be configured to augment the user's strength to both make using the prosthetic easier and to encourage them to use what strength they have.

The hybrid-drive prosthetic device 100 is illustrated and described with reference to FIGS. 1A through 1J. In embodiments, the hybrid-drive prosthetic device 100 includes at least a first (upper/proximal) arm portion 102 and a second (lower/distal) arm portion 104. The first arm portion 102 is configured to at least partially surround a portion of a limb. For example, the first arm portion 102 (sometimes referred to as an “arm gauntlet”) may be configured to at least partially surround a portion of a user's residual limb that is above or below the user's elbow. In some embodiments, at least one strap may be used to secure the first arm portion 102 to the upper (above-elbow) portion of the residual limb. The strap may employ VELCRO, a belt buckle, buttons, hooks, or any connector(s) to secure the strap to the residual limb. The second arm portion 104 is pivotally coupled to the first arm portion 102. For example, as shown in FIG. 1B, the second arm portion 104 may be connected to the first arm portion 102 by one or more hinges 103 (e.g., hinge 103A and hinge 103B) at an elbow joint of the hybrid-drive prosthetic device 100. Suitable pivots for hinges 103 may include, but are not limited to, pins, shafts, snap-fit concentric circular connectors, or any other type of pivot/hinge connectors that allow the second arm portion 104 to pivot toward the first arm portion 102 by flexing the elbow joint of the hybrid-drive prosthetic device 100.

The second arm portion 104 includes a socket 105 configured to receive the distal end of the user's residual limb (e.g., a distal end of the residual forearm in the case of a wrist or mid-forearm amputation). When the residual limb includes at least a portion of the user's forearm, the elbow joint of the hybrid-drive prosthetic device 100 may be actuated by flexing the user's actual elbow. In this regard, the second arm portion 104 may be configured to pivot toward the first arm portion 102 in response to elbow flexion of the residual limb.

A terminal device 106 (e.g., a prosthetic hand or any other end-effector(s)) may be coupled to a distal end of the second arm portion 104. In embodiments, the hybrid-drive prosthetic device 100 further includes one or more actuators 112 (e.g., actuators 112A and 112B) fixed to the first arm portion 102 and one or more cables 114 (e.g., cables 114A and 114B) extending from the one or more actuators 112 along one or more cable paths 116 (e.g., cable paths 116A and 116B) defined through the first arm portion 102 and the second arm portion 104 to the terminal device 106. For example, the cables 114A and 114B may extend from the actuators 114A and 114B through input openings 128A and 128B and output openings 130A and 130B in the first arm portion 102, then from the output openings 130A and 130B to input openings 132A and 132B in the second arm portion 104, and through the second arm portion 104 to the terminal device 106. Disposing the actuators 112A and 112B at a proximal portion of the hybrid-drive prosthetic device 100 (e.g., at the first arm portion 102) may have advantages over placement of actuators at or near the terminal device 106 because otherwise the terminal device 106 becomes heavy, making it difficult to carry heavy objects.

The cables 114A and 114B are held by or fed through the actuators 112A and 112B, respectively. For example, when the actuators 112A and 112B are linear actuators, the cables 114A and 114B may be held by held by or fed through openings 136A and 136B at distal ends of the actuator shafts 134A and 134B of the actuators 112A and 112B, respectively. Alternatively, the cables 114A and 114B may be held by, fed through, or secured to a rotating shaft, gear, or other moving member of the one or more actuators 112, which may include, but are not limited to, linear actuators, electric motors, and/or servos.

In some embodiments, one or more cable tensioners 126 are coupled to the one or more cables 114. For example, in FIG. 1B, the cables 114A and 114B are wound around a cable tensioner 126 (e.g., a BOA dial tensioner) configured to tighten or loosen the cables 114A and 114B. This allows for simplified calibration and/or adjustment of the cable system for the hybrid-drive prosthetic device 100. Although the embodiments in FIGS. 1A and 1B show a shared cable tensioner 126 for cables 114A and 114B; in other embodiments, the hybrid-drive prosthetic device 100 may include separate cables tensioners for each of the cables 114A and 114B.

The one or more cables 114 may be configured to control one or more components 110 (e.g., actuatable components/portions) of the terminal device 106. In some embodiments, the one or more cables 114 are directly coupled to the one or more components 110 of the terminal device 106. Alternatively, as shown in FIG. 1A, the terminal device 106 may include one or more secondary cables 118. The one or more secondary cables 118 may be coupled to the one or more cables 114 and configured to actuate the one or more components 110 of the terminal device 106 when the one or more cables 114 pull the one or more secondary cables 118 in response to being actuated by the one or more actuators 112 and/or when the second arm portion 104 is pivoted toward the first arm portion 102. In some embodiments, the one or more cables 114 are coupled to the one or more secondary cables 118 by a swivel connector to allow for partial or full rotation of the terminal device 106 relative to the second arm portion 104 without entanglement of cables 114 or cables 118. The swivel connector may be disposed or formed within in a simulated/prosthetic wrist 120 between the terminal device and the second arm portion 104. Alternatively, the swivel connector may be disposed within the terminal device 106 itself. For example, the swivel connector may be disposed within a support member 108 for the one or more actuatable components 110 of the terminal device 106.

In the embodiments illustrated in FIGS. 1A through 1J, the terminal device 106 is a hand and the one or more components 110 are fingers configured to be actuated by the one or more secondary cables 118. In this embodiment, the support member 108 is a palm of the hand. In an example implementation, the hand includes five fingers with two degrees of freedom. The finger and thumb may be oriented in opposition to facilitate cylindrical grasp and tip pinch. In some embodiments, silicone finger pads are added to provide increase friction for grasping activities. A rotation mechanism placed on the wrist 120 may be configured to permit full/partial pronation and supination, where the swivel connector allows for wrist rotation without twisting cables 114 and 118. In some embodiments, the rotation mechanism of the wrist 120 includes an inner circular disc/shaft with a center opening. A circle of embedded magnets with matching polarity may be placed around the disc. A bi-valve circular sleeve with embedded magnets aligned to match the disc magnets may be placed over the disc, where the magnets are placed with opposing polarity to assure mutual attraction. Consequently, the disc and sleeve rotate independently and are stabilized in various positions by the attraction of the magnets. In some embodiments, the magnets are sealed in a protective sleeve for safety.

The one or more cables 114 are configured to be actuated by the one or more actuators 112. For example, actuators 112A and 112B may be configured to actuate (e.g., pull/push, wind/unwind, or otherwise tension/loosen) cables 114A and 114B, respectively, in order to control movement of the one or more components 110 of the terminal device 106. The one or more cables 114 are further configured to be actuated by pivoting the second arm portion 104 toward the first arm portion 102. For example, when the second arm portion 104 is pivoted toward the first arm portion 102, the respective cable paths 116A and 116B for cables 114A and 114B are distorted (e.g., lengthened) as a result of increased distances between openings 130A and 132A and between openings 130B and 132B, and/or as a result of cable displacement by one or more protrusions 138 (e.g., protrusions 138A and 138B) that extend from the first arm portion 102. The one or more protrusions 138 may be configured to press or push upon the one or more cables 114 when the one or more cables 114 are actuated by pivoting the second arm portion 104 toward the first arm portion 102.

In some embodiments, the one or more protrusions 138 may be replaceable or adjustable to control an amount that the one or more cables 114 are actuated/displaced by pivoting the second arm portion 104 toward the first arm portion 102. For example, the one or more protrusions 138 may comprise removably coupled protruding members/leads that are disposed proximate to openings 130 and configured to extend distally from the first arm portion 102. The removably coupled protruding members may be selected from a plurality of protruding members with various dimensions that correspond to various amounts of cable displacement per angle of elbow flexion. In this regard, shorter protrusions 138 may be selected when greater elbow flexion is desired for body-powered actuation of the cables 114 and longer protrusions 138 may be selected when less elbow flexion is desired for body-powered actuation of the cables 114. Alternatively, or additionally, the protrusions 138 may comprise adjustable protruding members. For example, the protrusions 138 may be shortened/lengthened by employing a screw mechanism, ratchet mechanism, or the like.

In embodiments, the second arm portion 104 may be pivoted toward the first arm portion 102 when the user flexes the elbow portion of the hybrid-drive prosthetic device 100 (e.g., by flexing the user's actual elbow or otherwise causing the prosthetic elbow to flex). This allows for semi-automated (human-assisted/body-powered) actuation of components 110 in the terminal device 106 when the actuators 112 are not capable of supplying enough torque to complete a task on their own.

One notable issue with the existing trans-radial, body-powered, 3D printed prostheses is the difficulty in object manipulation and force production due to factors including short residual limb length, object manipulation height, muscle fatigue and excess trunk involvement. These factors have been reported by users and observed by the researchers as barriers to effective and long-term use of the prostheses. In some embodiments of the hybrid-drive prosthetic device 100, this limitation is overcome by incorporating simple, linear actuator-based variable tensioner system. By introducing an independently controlled tensioning component, both required range of motion and force production may be significantly enhanced. By placing the shafts 134A and 134B of the linear actuators 112A and 112B perpendicular to cable paths 116A and 116B, respectively, their travel linearly increases or decreases tension in the cables 114A and 114B. The reduced cable displacement required while the actuators 112A and 112B are extended leads to a lower required elbow and trunk range of motion to complete a gripping task, allowing for the relief of difficulties introduced by object manipulation height and excess trunk involvement. If the actuators 112A and 112B are activated during the act of gripping, the grip force on the object may be enhanced, thereby reducing torque requirements of the user (especially important for users with short residual limbs) and alleviating muscle fatigue.

To help minimize the bulkiness and optimize efficiency of the actuators 112A and 112B, the actuators 112A and 112B may be placed on the ventral side of the most proximal area of the hybrid-drive prosthetic device 100. In some embodiments, the actuators 112A and 112B are at least partially disposed within recesses or cavities formed in the upper/outer surface first arm portion 102. The actuator shafts 134A and 134B may be aligned perpendicular to the tension cable paths 116A and 116B, respectively, and placed so that, when fully retracted, the actuators 112A and 112B have no interference with the cables 114A and 114B. When extended, the actuator shafts 134A and 134B then exert force to shorten the cable paths 116A and 116B, effectively reducing required (body-powered) cable displacement and applying external force simultaneously.

In some embodiments, the actuators 112A and 112B include linear actuators (e.g., PQ12 linear actuators) that are capable of 40N at 6 mm/s with a 100:1 gear ratio. In experiments with PQ12 linear actuators, the stall current has been found to hover around 210 mA, while the active current draw without load has been experimentally found to be around 100 mA. With a full stroke length of 20 mm, the PQ12 linear actuators have been found capable of successfully controlling a prosthetic arm. However, it is noted that the parts and values provided herein are merely illustrative of example embodiments of the hybrid-drive prosthetic device 100, and the specific parts and values provided herein may be modified without departing from the scope of this disclosure. Accordingly, the examples provided herein shall not be construed as limitations of the invention unless otherwise specified in the claims.

In embodiments, the one or more actuators 112 (e.g., actuators 112A and 112B) are communicatively coupled to at least one controller 122 (e.g., microcontroller, microprocessor, programmable logic array, or the like). For example, the actuators 112A and 112B may be communicatively coupled to the controller 122 via communicative couplings 124A and 124B (e.g., wires, ribbon cables, flexible circuit boards, or the like). In some embodiments, the actuators 112A and 112B may be communicatively coupled to the controller 122 via wireless connectivity protocols (e.g., RF, Bluetooth, NFC, and/or optical communication interfaces).

The controller 122 may be configured to generate one or more signals that cause the one or more actuators 112 to actuate the one or more cables 114 in response to one or more sensor measurements received by the controller 122. For example, the controller 122 may be configured to detect user movements or pulses via myoelectric sensors, pressure/force sensors, optical sensors, electromagnetic sensors, or the like. Alternatively, the controller 122 may be coupled to a user-interface device (e.g., joystick, control pad, etc.), where the controller 122 may be configured to generate one or more signals that cause the one or more actuators 112 to actuate the one or more cables 114 in response to one or more user inputs received by the controller 122 via the user-interface device.

In some embodiments, the controller 122 may include a microcontroller (e.g., ATmega32U4 microcontroller) to allow for USB based firmware uploads and may be clocked at 8 MHz with an external crystal oscillator with a system voltage of 3.3V. The controller 122 may be paired with a wireless communication interface (e.g., HM-11 Bluetooth low Energy Module or any other wireless transceiver device) flashed with control software/firmware (MyoBridge firmware) to allow communication between the controller 122 and a myoclectric arm band (e.g., a Myo Armband) or any other sensors for detecting user movements/pulses. To enable portability, the hybrid-drive prosthetic device 100 may further include an energy storage device (e.g., a lithium-polymer battery) and integrated charging circuit. In some embodiments, the battery voltage being regulated down to an operating voltage (e.g., 3.3V) using a low-dropout (LDO) regulator (e.g., the AP2112K 600 mA LDO). Industry standard design considerations may be implemented including, for example, 0.1 uF bypass capacitors on digital and analog power supplies to suppress RF emissions as well as a secondary diode and 100 nF bypass capacitor on the reset line to suppress noise and protect the pin from overvoltage. In addition, series 220 resistors may be added onto USB differential lines to suppress ringing and 47 pF bypass capacitors added on the same lines for stability. It is noted that the parts and values provided herein are merely illustrative of example embodiments of the hybrid-drive prosthetic device 100; however, the specific parts and values provided herein may be modified without departing from the scope of this disclosure. Accordingly, the examples provided herein shall not be construed as limitations of the invention unless otherwise specified in the claims.

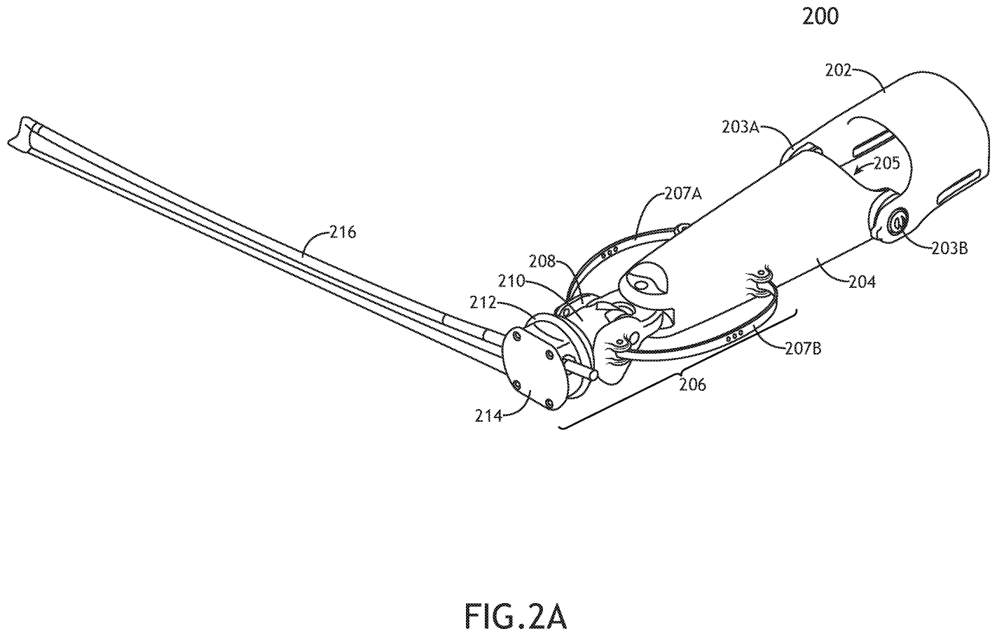

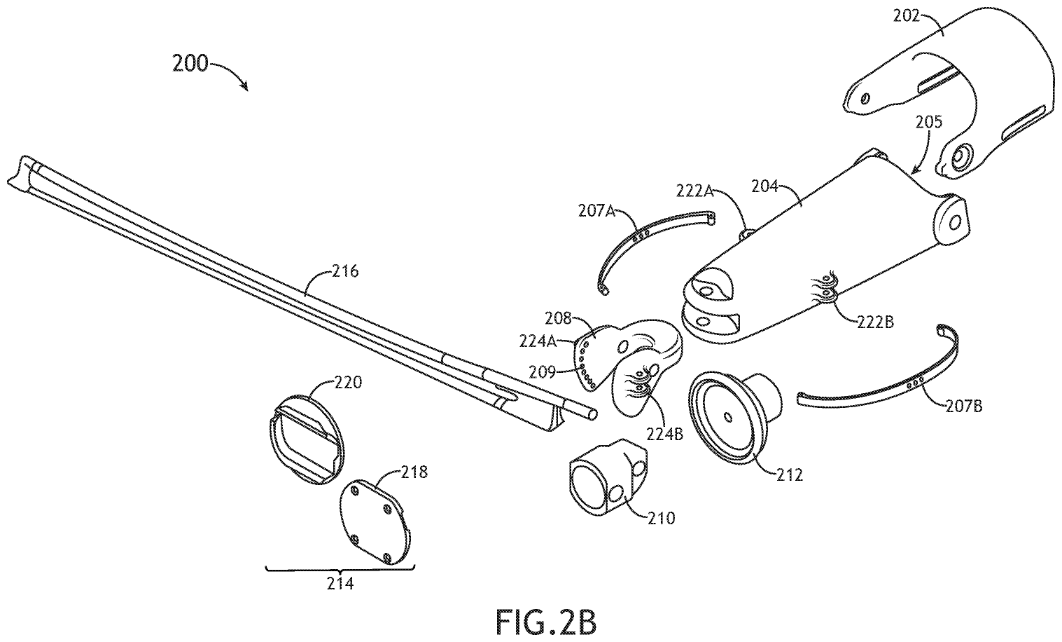

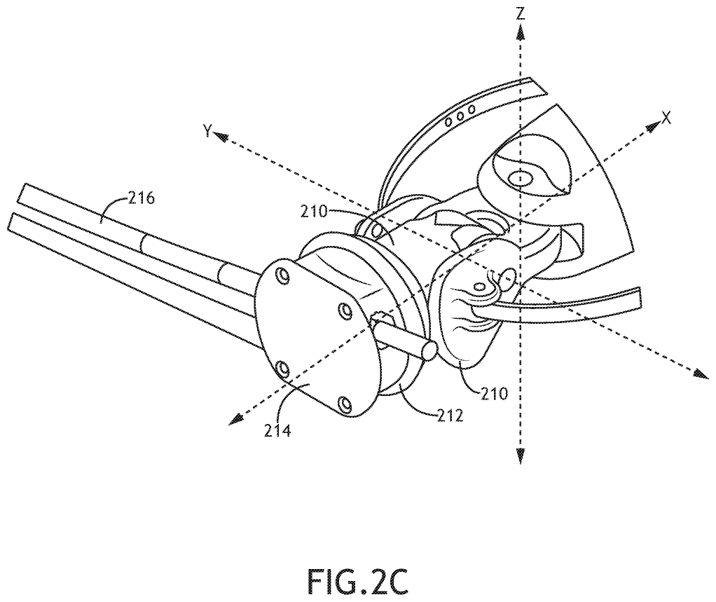

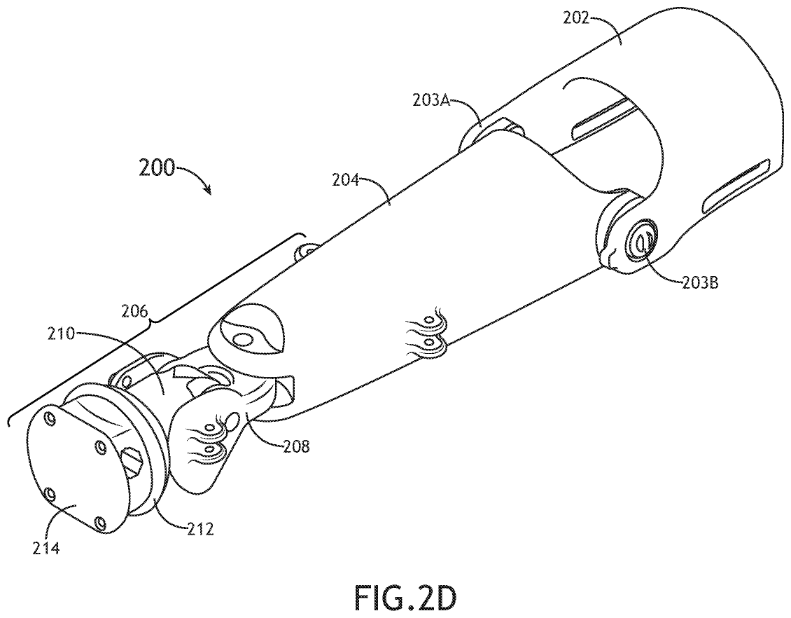

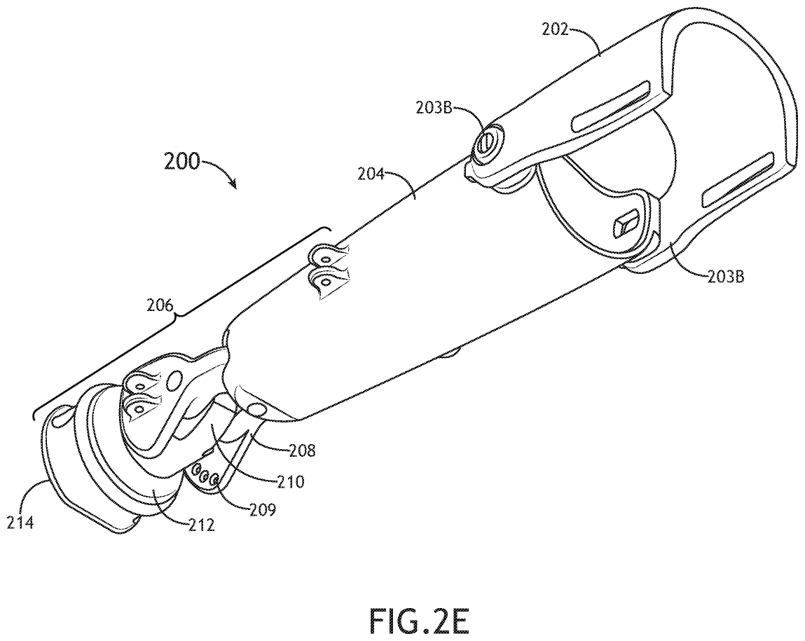

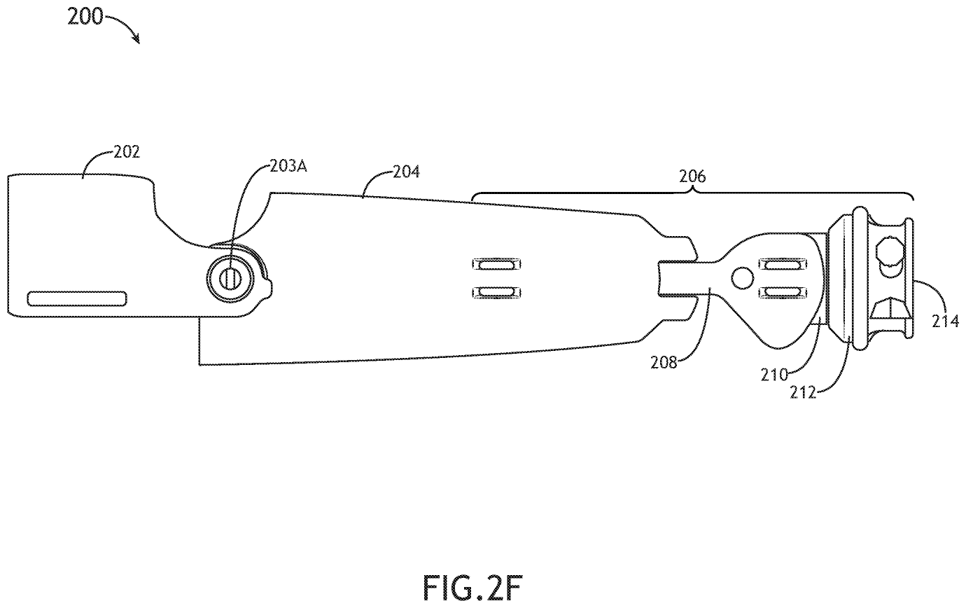

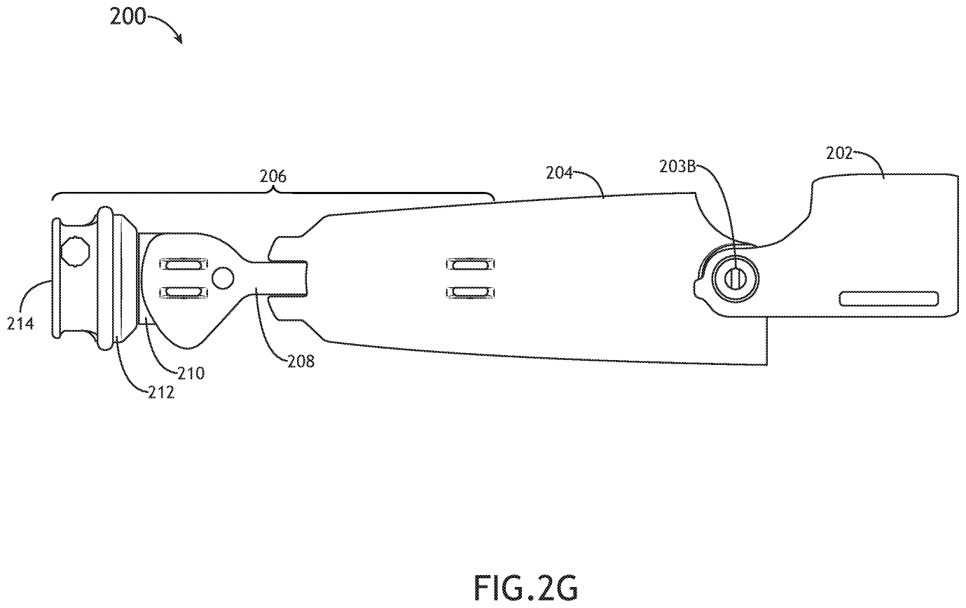

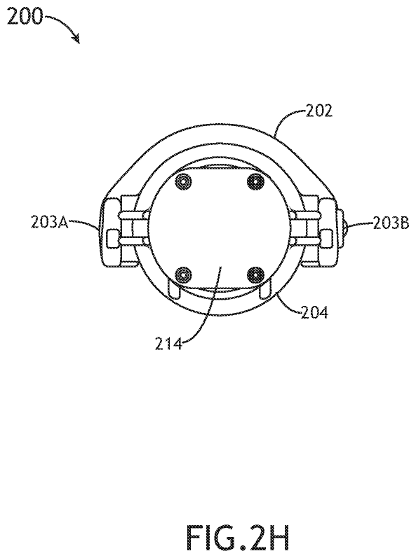

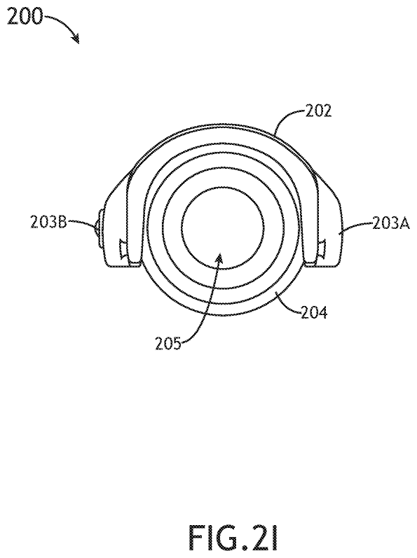

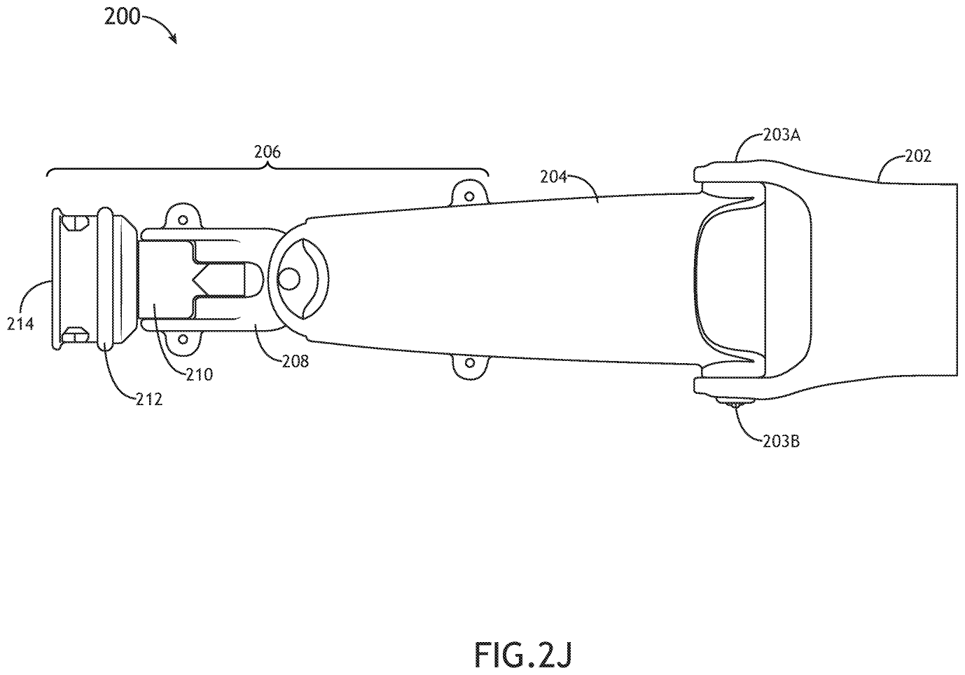

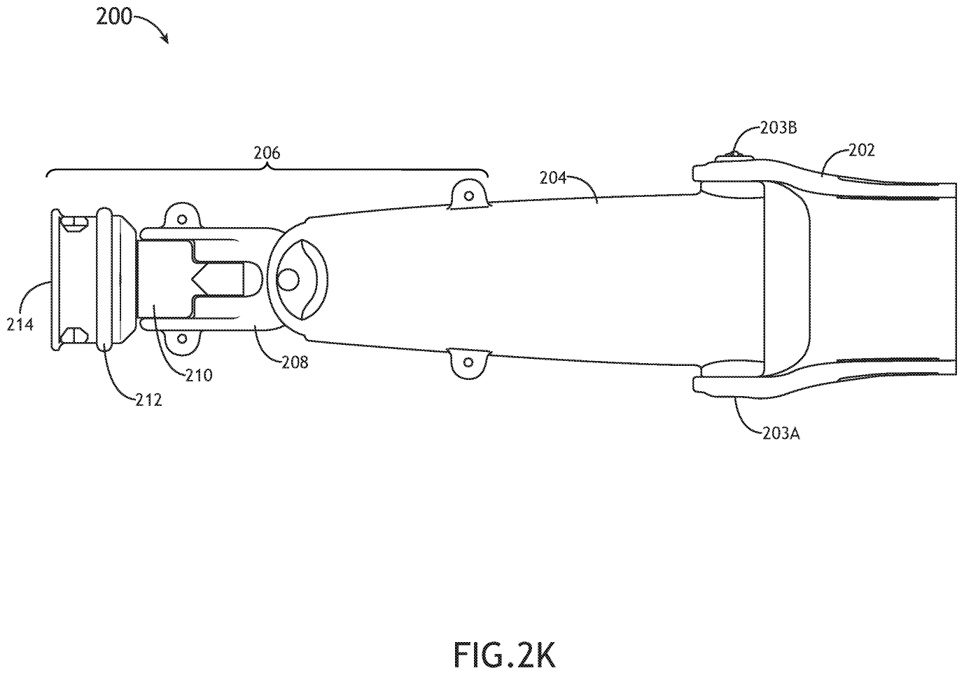

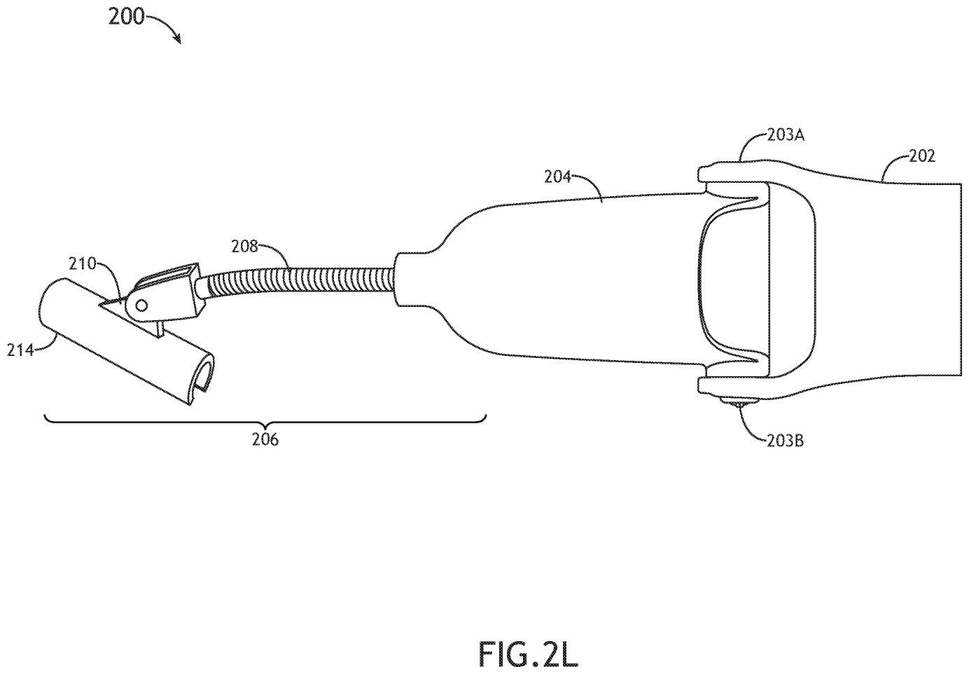

Referring now to FIGS. 2A through 2K, various embodiments of this disclosure are directed to a modular prosthetic device 200. Generalized body-powered upper limb prostheses are effective at gross movements, such as full-hand grasping, but are not robust in the performance of more morphologically or technically challenging tasks. For this reason, existing prostheses are generally not intended for specific activities. Additionally, those existing devices which are activity-specific cannot be easily adapted to other specific tasks. Consequently, there is also a need for upper limb prostheses with improved adaptability for specific tasks. The disclosed modular prosthetic device 200 allows for the use of a standardized prosthesis for a wide range of activities, far beyond the capabilities of existing body-powered prostheses, through the implementation of modular end-effectors that are optimized for use in varied situations. These modular end-effectors are simple to replace, lightweight, and inexpensive; this allows for customization of the prosthetic device's capabilities to match the demands of the user and the activity. The modular, easy-to-replace end effectors may be targeted for use in task-specific activities, including but not limited to, cello playing, violin playing, bike riding, swimming, pitching, basketball, football, golf, and so forth.

The modular prosthetic device 200 provides advantages over current existing prostheses which are activity-specific because those current devices cannot be easily adapted to other specific tasks. In embodiments of the modular prosthetic device 200, end effectors are mounted to a universal mating geometry located at the simulated/prosthetic wrist joint, and can introduce varied mechanisms for end effector control and manipulation (e.g., passive spring resistance, dashpots, tensioning cables, clamps, fasteners, etc.) in tasks requiring significantly different movement patterns and force production.

Existing prostheses tend to be bulky, expensive, and tailored to a single task or generalized to common grip patterns. Prosthetic devices which are generalized to common grip patterns (i.e. cylindrical grasp and palmar pinch) work well for gross manual tasks and basic object manipulation, but fail in the performance of tasks requiring sophisticated movements or modified kinematics. Alternative, task-specific devices may be used in these more complex and difficult movements, but often need to be completely donned/doffed by the user due to a lack of universally modular end effectors. The modular prosthetic device 200 differs from both of these options in that it is lightweight, low-cost and may be fitted with a large variety of compatible end effectors for various tasks. In this regard, the modular prosthetic device improves on pre-existing designs by simplifying the use of task-specific prosthetic devices, and creating a large set of end effectors which may be implemented quickly and simply, without the need of donning/doffing the prosthesis.

The modular prosthetic device 200 is illustrated and described with reference to FIGS. 2A through 2K. In embodiments, the modular prosthetic device 200 includes at least a first (upper/proximal) arm portion 202 and a second (lower/distal) arm portion 204. The first arm portion 202 is configured to at least partially surround a portion of a limb. For example, the first arm portion 202 (sometimes referred to as an “arm gauntlet”) may be configured to at least partially surround a portion of a user's residual limb that is above the user's elbow. In some embodiments, at least one strap may be used to secure the first arm portion 202 to the upper (above-elbow) portion of the residual limb. The strap may employ VELCRO, a belt buckle, buttons, hooks, or any connector(s) to secure the strap to the residual limb. The second arm portion 204 may be pivotally coupled to the first arm portion 202. For example, as shown in FIG. 2A, the second arm portion 204 may be connected to the first arm portion 202 by one or more hinges 203 (e.g., hinge 203A and hinge 203B) at an elbow joint of the modular prosthetic device 200. Suitable pivots for hinges 203 may include, but are not limited to, pins, shafts, snap-fit concentric circular connectors, or any other type of pivot/hinge connectors that allow the second arm portion 204 to pivot toward the first arm portion 202 by flexing the elbow joint of the modular prosthetic device 200. In other embodiments, the modular prosthetic device 200 may include only one arm portion (e.g. arm portion 204), or the first arm portion 202 and the second arm portion 204 may be rigidly connected instead of being pivotally connected, depending on the amputation and/or based the task-specific end effector being employed.

The second arm portion 204 includes a socket 205 configured to receive the distal end of the user's residual limb (e.g., a distal end of the residual forearm in the case of a wrist or mid-forearm amputation). When the residual limb includes at least a portion of the user's forearm, the elbow joint of the modular prosthetic device 200 may be actuated by flexing the user's actual elbow. In this regard, the second arm portion 204 may be configured to pivot toward the first arm portion 202 in response to elbow flexion of the residual limb.

In embodiments, a modular terminal device assembly 206 is removably coupled to a distal end of the second arm portion 204. As shown in FIG. 2A, the modular terminal device assembly 206 includes at least a first modular link 208, a second modular link 210, and a third modular link 212. FIG. 2B is an exploded view of the modular prosthetic device 100 illustrating the individual components (e.g., the first modular link 208, second modular link 210, and third modular link 212) of the modular terminal device assembly 206.

As shown in FIGS. 2A and 2B, the modular terminal device assembly 206 may include an end effector 216 (e.g., a cello bow) configured to be coupled to the third modular link 212 by an attachment device 214 that includes support members 218 and 220 that are secured/securable to the third modular link 212. The support members 218 and 220 may be configured to receive the end effector 216 in between the support members 218 and 220 so that the end effector 216 is secured to the third modular link 212 by the support members 218 and 220. In some embodiments, the support members 218 and 220 are fastened to the third modular link 212 by elastic cables (e.g., elastomeric TPU cables) and clamps the end effector 216 securely for performance of the task.

In other embodiments, the attachment device 214 may include, but is not limited to, clamp(s), cooperatively threaded connector(s), snap-fit connector(s), elastic sleeve(s), pin(s), screw(s), ratcheting connector(s), ball-and-socket connector(s), or the like. Furthermore, the attachment device 214 may be configured to removably couple the end effector 216 to any of the modular links (e.g., the first modular link 208, second modular link 210, or third modular link 212) or to the distal end of the second arm portion 204 itself. In some embodiments, the attachment device 214 may also be integrated within the end effector 216. For example, the attachment device 214 may be formed or disposed at a proximal end of the end effector 216.

Referring now to FIG. 2C, the first modular link 208 is removably coupled to the second arm portion 204 and is configured to pivot about a first axis (the “Z-axis”) to control yaw (e.g., abduction/adduction or radial/ulnar deviation) of the end effector 216. The second modular link 210 is removably coupled to the first modular link 208 and is configured to pivot about a second axis (the “Y-axis”) to control pitch (e.g., flexion/extension) of the end effector 216. And the third modular link 212 is removably coupled to the second modular link 210 and is configured to rotate about a third axis (the “X-axis”) to control roll (e.g., pronation/supination) of the end effector 216. In the embodiment illustrated in FIG. 2C, the first axis, the second axis, and the third axis are orthogonal. However, in other embodiments, one or more of the axes may be non-orthogonal with respect to one another.

As shown in FIGS. 2A and 2B, the first modular link may be pivotally coupled to the second arm portion 204 at a hinge. The modular prosthetic device 200 may further include one or more leaf springs 207 (e.g., leaf springs 207A and 207B) coupled to the first modular link 208 (e.g., at connection points 224A and 224B) and to the second arm portion 204 (e.g., at connection points 222A and 222B). The leaf springs 207A and 207B can be configured to bias the first modular link 208 to a default position in absence of another pivotal force acting on the first modular link 208. In this regard, the first modular link 208 may be configured to return to a default position after being pivoted clockwise or counterclockwise about the Z-axis for abduction/adduction of the end effector 216. In other embodiments, a plurality of spring-loaded ball detents at the hinge between the first modular link 208 and second arm portion 204 may define discrete points along which the first modular link 208 is configured to pivot about the Z-axis. In such embodiments, the first modular link 208 may be pivoted and temporarily set at one of the discrete points. The spring-loaded ball detents may be located on the second arm portion 204 and configured to mate with features (e.g., dimples/holes) on the first modular link 208, or vice versa.

The second modular link 210 may be pivotally coupled to the first modular link 208 at a hinge. The modular prosthetic device 200 may further include a plurality of spring-loaded ball detents 209 at the hinge between the first modular link 208 and second modular link 210. The spring-loaded ball detents 209 may define discrete points along which the second modular link 210 is configured to pivot about the Y-axis for flexion/extension of the end effector 216. In this regard, the second modular link 210 may be pivoted and temporarily set at one of the discrete points. The spring-loaded ball detents 209 may be located on the first modular link 208 and configured to mate with features (e.g., dimples/holes) on the second modular link 210, or vice versa.

The third modular link 212 may be coupled to the second modular link 210 and includes one or more portions configured to rotate with respect to the second modular link 210. In this manner, the third modular link 212 allows the end effector 216 to rotate about the X-axis for pronation/supination of the end effector 216. The modular prosthetic device 200 may further include at least one torsion spring configured to bias the third modular link 212 to a default position in absence of another rotational force acting on the third modular link 212. For example, the third modular link 212 may include a torsion spring disposed between a rotatable portion of the third modular link 212 and a base structure and/or between the rotatable portion of the third modular link 212 and the second modular link 210. In this regard, the third modular link 212 may be configured to return to a default position after being rotated clockwise or counterclockwise about the X-axis for pronation/supination of the end effector 216. In other embodiments, the modular prosthetic device 200 may include a plurality of spring-loaded ball detents between a rotatable portion of the third modular link 212 and a base structure and/or between the rotatable portion of the third modular link 212 and the second modular link 210. The spring-loaded ball detents may define discrete points along which the third modular link 212 is configured to rotate about the X-axis. In such embodiments, the third modular link 212 may be rotated and temporarily set at one of the discrete points. The spring-loaded ball detents may be located on the rotatable portion of the third modular link 212 and configured to mate with features (e.g., dimples/holes) on the base structure, or vice versa. Alternatively, the spring-loaded ball detents may be located on the rotatable portion of the third modular link 212 and configured to mate with features (e.g., dimples/holes) on the second modular link 210, or vice versa.

The third modular link 212 may be configured to be coupled to a first type of end effector 216 with six degrees of freedom (e.g., up/down, left/right, forward/backward, pitch, yaw, and roll). However, one or more of the modular links (e.g., the first modular link 208, second modular link 210, and/or third modular link 212) may be removed (e.g., detached) from the modular terminal device assembly 206 to allow for utilization of different end effectors 216 with differing degrees of freedom. For example, in an implementation, the third modular link 212 may be removed to couple a second type of end effector 216 with five degrees of freedom (e.g., up/down, left/right, forward/backward, pitch, and yaw) to the second modular link 210. In another example implementation, the second modular link 210 is also removed to couple a third type of end effector 216 with four degrees of freedom (e.g., up/down, left/right, forward/backward, and pitch) to the first modular link 208. In some implementations, all the modular links (e.g., the first modular link 208, second modular link 210, and the third modular link 212) may be removed. For example, the first modular link 208 may also be removed to couple a fourth type of end effector with three degrees of freedom (e.g., up/down, left/right, and forward/backward) directly to the second arm portion 204.

The end-effector 116 may include, but is not limited to, a prosthetic hand, a tool (e.g., a hammer, a saw, a screwdriver, a chisel, a mallet, a hatchet, a hook, a claw, etc.), an instrument (e.g., a bow, a drumstick, a violin, a flute, a trumpet, etc.), sports gear (e.g., a bat, a club, a racquet, a hockey stick, a polo stick, a mitt, etc.), hunting gear (e.g., a rifle, a pellet gun, a slingshot, an archery bow, etc.), or any other type of general or task-specific handheld device. Depending on the motion and stability/rigidity requirements of the end effector 216, the modular terminal device assembly 206 is modified by adding/removing modular links. For example, less of the modular links and hence less freedom of motion may be appropriate when more stability/rigidity is required. Alternatively, more freedom of motion and hence more of the modular links may be appropriate when less stability/rigidity is required.

In an example implementation, where the end effector 216 is a cell bow, once the modular prosthetic device 200 is fitted to the user's residual limb, passive control of the cello bowing position is accomplished via spring tension regulation of wrist adduction/abduction by leaf springs 207A and 207B and the first (adductor/abductor) modular link 208. Wrist flexion may be user-selectable with 5 to 900 adjustment angles through the use of spring-loaded ball detents in the second (flexor) modular link 210 which engages with detents 209 in the first (adductor/abductor) modular link 208. Maintenance of force of the cello bow on the instrument's strings may be performed by a small compression/torsion spring located in the third (pronator/supinator) modular link 212. Secure fixturing of the end effector 216 (e.g., the cello bow) may be accomplish by an attachment device 214 that is secured to the third modular link 212. In some embodiments, the attachment device 214 secures the end effector 216 to a rotatable portion of third modular link 212 by holding the end effector 216 between support members 218 and 220 that are held together by elastomeric cable.

Another embodiment of the modular prosthetic device 200 is illustrated in FIG. 2L, where the first modular link 208 is a flexible arm capable of controlling pitch, raw, and roll, at least to some degree. In this embodiment, the second modular link 210 is integrated with the attachment device 214. However, the second modular link 210 and the attachment device 214 can alternatively be separate components. In the example illustrated in FIG. 2L, the attachment device 214 is a holder configured to receive an end effector 216 (e.g., a golf club) such that the flexible arm (modular link 208) supports the end effector 216 in a relatively rigid manner while still providing resiliency to withstand impacts encountered by the end effector 216 (e.g., golf club hitting a golf ball, or the like).

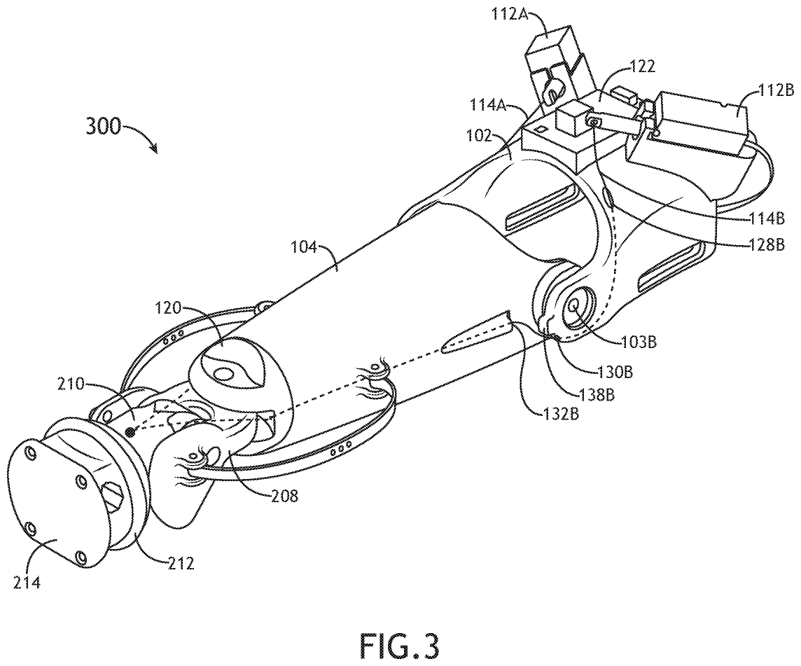

Embodiments of a hybrid-drive prosthetic device 100 have been described with reference to FIGS. 1A through 1J, and embodiments of the modular prosthetic device 200 have been separately described with reference to FIGS. 2A through 2L. However, additional embodiments may combine aspects of the hybrid-drive prosthetic device 100 and the modular prosthetic device 200. For example, FIG. 3 illustrates an embodiment of a hybrid-drive modular prosthetic device 300.

As shown in FIG. 3, the hybrid-drive modular prosthetic device 300 may include the same or substantially the same structure as the hybrid-drive prosthetic device 100 up to the wrist 120 location of the second arm portion 104. Then, instead of the terminal device 106, the hybrid-drive modular prosthetic device 300 may include the same or substantially same structure as the modular terminal device assembly 206 of the modular prosthetic device 200. In this regard, the hybrid-drive modular prosthetic device 300 may include one or more actuators 112 (e.g., actuators 112A and 112B) fixed to the first arm portion 102 and one or more cables 114 (e.g., cables 114A and 114B) extending from the one or more actuators 112 along one or more cable paths defined through the first arm portion 102 and the second arm portion 104 to the modular terminal device assembly 206. For example, the cables 114A and 114B may extend from the actuators 114A and 114B through input openings 128A and 128B and output openings 130A and 130B in the first arm portion 102, then from the output openings 130A and 130B to input openings 132A and 132B in the second arm portion 104, and through the second arm portion 104 to the modular terminal device assembly 206.

The one or more cables 114 may be configured to control one or more components (e.g., the first modular link 208, second modular link 210, third modular link 212, and/or end effector 216) of the modular terminal device assembly 206. For example, in the embodiment illustrated in FIG. 3, the cables 114A and 114B are connected to the second modular link 210 for controlling pitch or flexion/extension of the end effector 216. The one or more cables 114 are configured to be actuated by the one or more actuators 112. For example, actuators 112A and 112B may be configured to actuate (e.g., pull/push, wind/unwind, or otherwise tension/loosen) cables 114A and 114B, respectively, in order to control movement of the one or more (e.g., the first modular link 208, second modular link 210, third modular link 212, and/or end effector 216) of the modular terminal device assembly 206. The one or more cables 114 are further configured to be actuated by pivoting the second arm portion 104 toward the first arm portion 102. For example, when the second arm portion 104 is pivoted toward the first arm portion 102, the respective cable paths for cables 114A and 114B are distorted (e.g., lengthened) as a result of increased distances between openings 130A and 132A and between openings 130B and 132B, and/or as a result of cable displacement by one or more protrusions 138 (e.g., protrusions 138A and 138B) that extend from the first arm portion 102. The one or more protrusions 138 may be configured to press/push upon the one or more cables 114 when the one or more cables 114 are actuated by pivoting the second arm portion 104 toward the first arm portion 102.

In some embodiments, the one or more protrusions 138 may be replaceable or adjustable to control an amount that the one or more cables 114 are actuated/displaced by pivoting the second arm portion 104 toward the first arm portion 102. For example, the one or more protrusions 138 may comprise removably coupled protruding members/leads that are disposed proximate to openings 130 and configured to extend distally from the first arm portion 102. The removably coupled protruding members may be selected from a plurality of protruding members with various dimensions that correspond to various amounts of cable displacement per angle of elbow flexion. In this regard, shorter protrusions 138 may be selected when greater elbow flexion is desired for body-powered actuation of the cables 114 and longer protrusions 138 may be selected when less elbow flexion is desired for body-powered actuation of the cables 114. Alternatively, or additionally, the protrusions 138 may comprise adjustable protruding members. For example, the protrusions 138 may be shortened/lengthened by employing a screw mechanism, ratchet mechanism, or the like.

In embodiments, the second arm portion 104 may be pivoted toward the first arm portion 102 when the user flexes the elbow portion of the hybrid-drive modular prosthetic device 300 (e.g., by flexing the user's actual elbow or otherwise causing the prosthetic elbow to flex). This allows for semi-automated (human-assisted/body-powered) actuation of components (e.g., the first modular link 208, second modular link 210, third modular link 212, and/or end effector 216) of the modular terminal device assembly 206 when the actuators 112 are not capable of supplying enough torque to complete a task on their own.

Various components (e.g., first arm portion 102/202, second arm portion 104/204, terminal device 106/modular terminal device assembly 206 components, etc.) of the prosthetic devices 100, 200, and 300 described herein may be produced by additive manufacturing processes (e.g., 3D printing). In addition to making the prostheses more customizable, 3D printing may allow for reduced cost and better user-experience, particularly when working with children.

The most common 3D printing method for 3D printed prostheses is fused deposition modeling. Fused deposition modeling is a form of additive manufacturing that involves melting thin layers of plastic over each other to form a 3D structure. The two most common 3D printed filament materials used to manufacture upper limb prostheses is polylactide filament and acrylonitrile butadiene styrene filament. Polylactide filament has similar properties to a thermoplastic and permits minor modifications through targeted heating once the device has been 3D printed. Acrylonitrile butadiene styrene filament however, does not offer homogenous thermoplastic properties making post-processing modifications difficult. Thus, the preferred material for 30 printed prostheses is polylactide filament given the ability to perform postprocessing modifications that may be required during assembly or clinical fitting.

After selecting the printing material, the files need to be sliced and nested before 3D printing. Slicing may be performed using open-source software. Slicing is the process that converts 3D models into a format understood by 30 printers. In some implementations, the model is “sliced” into 20 cross-sectional layers which may be placed by the extruder. The slicing software may develop a G-code which is a set of numerical values representing positions in the system-based reference frame in which the extruder needs to follow to trace and recreate the design. Furthermore, nesting is the process of organizing the printed parts in the building platform in an effective and efficient manner. Most consumer desktop 3D printers provide access to software that allows the user to set the preferred parameters. These parameters include percentage of infill and pattern (i.e., hexagon pattern), print speed (mm/s), temperature of the heated bed (° F./C), shell thickness (mm) and the option of using rafts and supports to ensure printing feasibility. 3D printed prostheses may be manufactured using low-cost desktop 3D printers (e.g., Ultimaker 2, Ultimaker B.V., Geldermalsen, The Netherlands) or industrial grade 3D printers (e.g., Uprint SE Plus, Stratasys, Minn., USA). When using desktop 3D printers, in some implementations, the settings are approximately 40% infill (hexagon pattern), 35 mm/s print speed, 150 mm/s travel speed, 65° C. heated bed for acrylonitrile butadiene styrene filament (room temperature for polylactide filament), 0.15 mm layer height, and 1 mm shell thickness. Rafts and supports may be utilized to avoid misprints. Furthermore, the application of light adhesive (e.g., glue stick) or painter's tape over the building platform of desktop 3D printers can aid in improving the adherence of the first plastic layer and facilitates removal of the printed structure. For industrial 3D printers, this may not be necessary since the building tray is often disposable and provides sufficient adherence. Due to the inherent anisotropic characteristics of fused deposition modeling, orientation of the different 3D printed prosthesis components over the building tray can play an important role in the durability of the prosthesis. As characteristics and mechanical properties of 3D printed parts are dependent on printing orientation, it is important to consider the direction of operational loads on each component. 3D printed parts using fused deposition modeling are much more likely to delaminate and fracture when placed in tension in the direction of build height compared to the orthogonal axes. For this reason, the 3D printed prostheses may be printed using a horizontal axis. There are only few exceptions to this rule specific to the layer height and dimension of the printed part. Lower layer height (higher resolution) typically results in a part being printed with smoother surfaces and less likelihood to delaminate or fracture. The duration of the print depends on the size of the prostheses, infill, layer height (resolution) and orientation of the printed part. For example, the printing time of a partial hand prosthesis for a 12-year old child may be between 6 and 8 hours.

It is noted that the parts, tools, and values provided herein are merely illustrative of example systems/processes for manufacturing the prosthetic devices 100, 200, and 300 described herein; however, the specific parts, tools, and values provided herein may be modified without departing from the scope of this disclosure. Accordingly, the examples provided herein shall not be construed as limitations of the invention unless otherwise specified in the claims.

Although the technology has been described with reference to the embodiments illustrated in the attached drawing figures, equivalents may be employed and substitutions made herein without departing from the scope of the technology as recited in the claims. Components illustrated and described herein are examples of devices and components that may be used to implement the embodiments of the present invention and may be replaced with other devices and components without departing from the scope of the invention. Furthermore, any dimensions, degrees, and/or numerical ranges provided herein are to be understood as non-limiting examples unless otherwise specified in the claims.

What is claimed is:

1. A hybrid-drive prosthetic device, comprising:
a first arm portion configured to at least partially surround a portion of a limb;
a second arm portion pivotally coupled to the first arm portion, the second arm portion including a socket configured to receive a distal end of the portion of the limb;
a terminal device coupled to the second arm portion;
one or more actuators fixed to the first arm portion; and
one or more cables for controlling one or more components of the terminal device, the one or more cables extending from the one or more actuators along one or more cable paths defined through the first arm portion and the second arm portion to the terminal device, the one or more cables configured to be actuated by the one or more actuators and further configured to be actuated by pivoting the second arm portion toward the first arm portion.

2. The hybrid-drive prosthetic device of claim 1, further comprising:
one or more secondary cables in the terminal device, wherein the one or more secondary cables are coupled to the one or more cables and configured to actuate the one or more components of the terminal device when the one or more cables pull the one or more secondary cables in response to being actuated by the one or more actuators or by pivoting the second arm portion toward the first arm portion.

3. The hybrid-drive prosthetic device of claim 2, further comprising:
a swivel connector coupling the one or more secondary cables to the one or more cables.

4. The hybrid-drive prosthetic device of claim 2, wherein the terminal device comprises a hand and the one or more components comprise fingers configured to be actuated by the one or more secondary cables.

5. The hybrid-drive prosthetic device of claim 1, wherein the one or more actuators comprise one or more linear actuators, electric motors, or servos.

6. The hybrid-drive prosthetic device of claim 1, wherein the one or more actuators comprise at least a first actuator and a second actuator fixed to the first arm portion, and the one or more cables comprise at least a first cable controlled by the first actuator and a second cable controlled by the second actuator.

7. The hybrid-drive prosthetic device of claim 1, further comprising:
one or more cable tensioners coupled to the one or more cables.

8. The hybrid-drive prosthetic device of claim 1, further comprising:
one or more protrusions extending from the first arm portion and configured to press upon the one or more cables when the one or more cables are actuated by pivoting the second arm portion toward the first arm portion.

9. The hybrid-drive prosthetic device of claim 8, wherein the one or more protrusions are replaceable or adjustable to control an amount that the one or more cables are actuated by pivoting the second arm portion toward the first arm portion.

10. The hybrid-drive prosthetic device of claim 1, further comprising:
one or more controllers communicatively coupled to the one or more actuators, the one or more controllers configured to generate one or more signals that cause the one or more actuators to actuate the one or more cables in response to one or more sensor measurements received by the one or more controllers.

11. A modular prosthetic device, comprising:
a first arm portion configured to at least partially surround a portion of a limb;
a second arm portion pivotally coupled to the first arm portion, the second arm portion including a socket configured to receive a distal end of the portion of the limb; and
a modular terminal device assembly removably coupled to the second arm portion, the modular terminal device assembly including:
a first modular link removably coupled to the second arm portion, the first modular link configured to pivot about a first axis to control yaw of an end effector;
a second modular link removably coupled to the first modular link, the second modular link configured to pivot about a second axis to control pitch of the end effector; and
a third modular link removably coupled to the second modular link, the third modular link configured to rotate about a third axis to control roll of the end effector.

12. The modular prosthetic device of claim 11, wherein the first axis, the second axis, and the third axis are orthogonal.

13. The modular prosthetic device of claim 11, further comprising:
one or more leaf springs coupled to the first modular link and the second arm portion, the one or more leaf springs configured to bias the first modular link to a default position in absence of another pivotal force acting on the first modular link.

14. The modular prosthetic device of claim 11, further comprising:
a plurality of spring-loaded ball detents between the second modular link and the first modular link, wherein the plurality of spring-loaded ball detents define discrete points along which the second modular link is configured to pivot about the second axis.

15. The modular prosthetic device of claim 11, further comprising:
at least one torsion spring configured to bias the third modular link to a default position in absence of another rotational force acting on the third modular link.

16. The modular prosthetic device of claim 11, wherein the third modular link is configured to be coupled to a first type of end effector with six degrees of freedom.

17. The modular prosthetic device of claim 16, wherein the third modular link is removable to couple a second type of end effector with five degrees of freedom to the second modular link.

18. The modular prosthetic device of claim 17, wherein the second modular link is removable to couple a third type of end effector with four degrees of freedom to the first modular link.

19. The modular prosthetic device of claim 18, wherein the first modular link is removable to couple a fourth type of end effector with three degrees of freedom to the second arm portion.

20. A hybrid-drive modular prosthetic device, comprising:
a first arm portion configured to at least partially surround a portion of a limb;
a second arm portion pivotally coupled to the first arm portion, the second arm portion including a socket configured to receive a distal end of the portion of the limb;
a modular terminal device assembly coupled to the second arm portion, the modular terminal device assembly including:
a first modular link removably coupled to the second arm portion, the first modular link configured to pivot about a first axis to control yaw of an end effector;
a second modular link removably coupled to the first modular link, the second modular link configured to pivot about a second axis to control pitch of the end effector; and
a third modular link removably coupled to the second modular link, the third modular link configured to rotate about a third axis to control roll of the end effector;
one or more actuators fixed to the first arm portion; and
one or more cables for controlling one or more components of the modular terminal device assembly, the one or more cables extending from the one or more actuators along one or more cable paths defined through the first arm portion and the second arm portion to the modular terminal device assembly, the one or more cables configured to be actuated by the one or more actuators and further configured to be actuated by pivoting the second arm portion toward the first arm portion.
